(19) United States
(12) Patent Application Publication
(10) Pub. No.: US 2008/0011307 A1
(43) Pub. Date: Jan. 17, 2008

(54) Hand assisted laparoscopic device

(76) Inventors: Andrew T. Beckman, Cincinnati, OH (US); Michael D. Cronin, Cincinnati, OH (US)

Correspondence Address: WELSH & FLAXMAN LLC, 2000 DUKE STREET, SUITE 100, ALEXANDRIA, VA, 22314, US

(21) Appl. No.: 11/484,846

(22) Filed: Jul. 12, 2006

(51) Int. Cl. A61B 19/08

(52) U.S. Cl. 128/850

(57) ABSTRACT

A hand assisted laparoscopic seal apparatus includes an outer seal, an inner seal and a housing supporting the outer seal relative to the inner seal, wherein one of the outer seal or the inner seal is shaped and dimensioned for engaging and sealing around an arm of a medical practitioner and the other seal is shaped and dimensioned for sealing a body cavity from an external environment.

### BACKGROUND OF THE INVENTION

1. Field of the Invention

The invention relates to laparoscopic devices. In particular, the invention relates to a laparoscopic seal assembly permitting the use of hands in assisting during laparoscopic procedures.

2. Description of the Prior Art

During laparoscopic procedures, it is often desirable for the surgeon to place his or her hand within the patient in a manner manipulating the instruments positioned within the patient. When this occurs, it is desirable to separate the external environment from the internal portion of the patient. For example, when hand assisted laparoscopic procedures are performed within the abdominal cavity, it is desirable to perform hand exchanges with minimal loss of abdominal pressure.

As such, a need exists for skin mountable seals permitting hand assisted laparoscopic procedures without fear that the abdominal pressure will be compromised. The present invention provides such an apparatus.

### SUMMARY OF THE INVENTION

It is, therefore, an object of the present invention to provide a hand assisted laparoscopic seal apparatus. The apparatus includes a first seal, a second seal and a housing supporting the first seal relative to the second seal. One of the first seal or the second seal is shaped and dimensioned for engaging and sealing around an arm of a medical practitioner and the other seal is shaped and dimensioned for sealing a body cavity from an external environment.

It is also an object of the present invention to provide a seal apparatus wherein the first seal is a lip seal having a central opening.

It is also another object of the present invention to provide a seal apparatus wherein the second seal is a duckbill seal.

It is also a further object of the present invention to provide a seal apparatus wherein the first seal is an outer seal and the second seal is an inner seal.

It is another object of the present invention to provide a seal apparatus wherein the lip seal includes a flexible annular membrane with the central opening formed therein.

It is a further object of the present invention to provide a seal apparatus wherein the central opening is approximately 2.00 inches or less.

It is yet a further object of the present invention to provide a seal apparatus wherein the central opening ranges between approximately 1.25 inches to approximately 1.5 inches.

It is still another object of the present invention to provide a seal apparatus wherein the first seal is an overlapping seal having a central opening.

It is also an object of the present invention to provide a seal apparatus wherein the second seal includes first and second seal bodies, each of the first and second seal bodies include an upper surface and a lower surface, and the respective first and second seal bodies includes a thickened section adjacent a meeting point of the first and second seal bodies.

It is another object of the present invention to provide a seal apparatus wherein the thickness of the first and second seal bodies is approximately 0.540 inch along the thickened section.

It is a further object of the present invention to provide a seal apparatus wherein the first and second seal bodies are approximately less than 0.10 inch thick along a remainder thereof.

It is also an object of the present invention to provide a seal apparatus including a seal and a housing supporting the seal, wherein the seal is composed of open cell foam impregnated with silicone.

It is still another object of the present invention to provide a seal apparatus wherein the seal is manufactured by casting the open cell foam within a mold and then pouring silicone therein to produce seal bodies exhibiting softness and durability.

It is yet a further object of the present invention to provide a seal apparatus wherein the open cell foam is polyurethane, urethane-ether or polyolefin.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of the present seal apparatus adjacent an incision in accordance with a preferred embodiment of the present invention.

FIG. 2 is a perspective view of the seal apparatus shown in FIG. 1 mounted at a predetermined tissue location.

FIG. 3 is a bottom perspective view of the inner seal of the seal apparatus shown with reference to FIG. 1.

FIG. 4 is a partially exploded view of the seal apparatus shown in FIG. 1.

FIG. 5 is an exploded view of the seal apparatus shown in FIG. 1.

FIG. 6 is a cross sectional view of the seal apparatus shown in FIG. 1.

FIGS. 7 and 8 are cross sectional views showing use of the seal apparatus shown in FIG. 1.

FIG. 9 is a perspective view of the present seal apparatus in accordance with an alternate embodiment of the present invention.

FIG. 10 is an exploded view of the seal apparatus shown in FIG. 9.

FIGS. 11 and 12 are cross sectional views showing use of the seal apparatus shown in FIG. 9.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for teaching one skilled in the art how to make and/or use the invention.

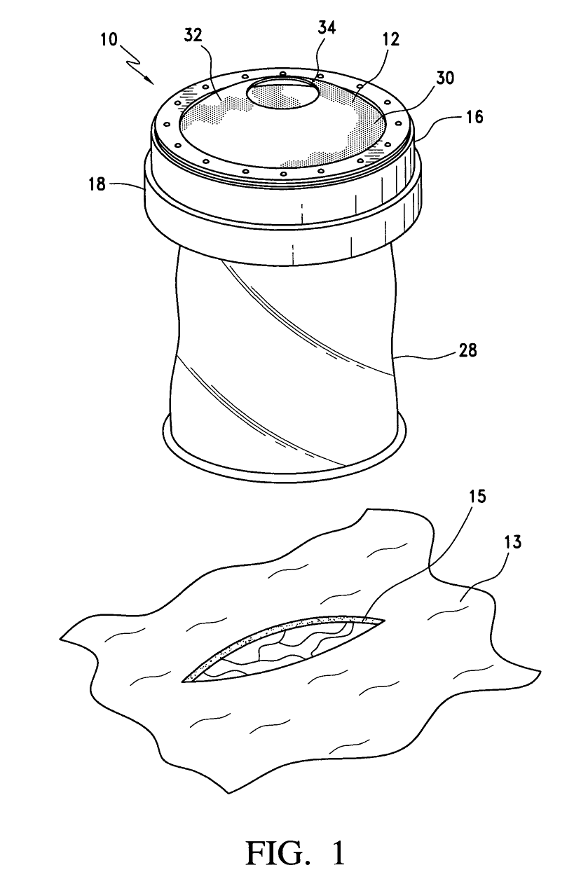

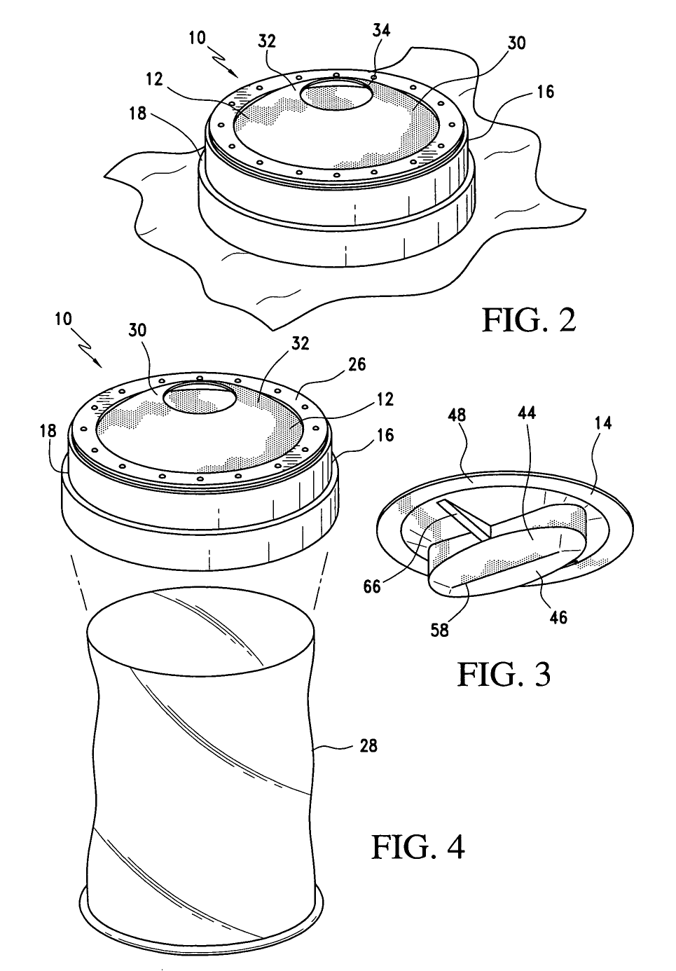

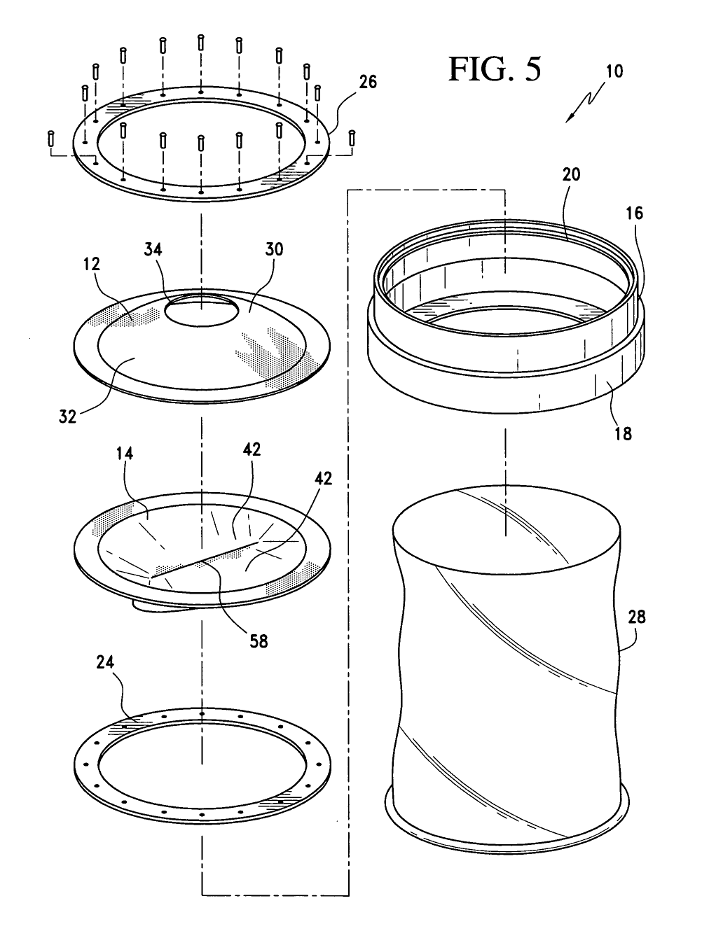

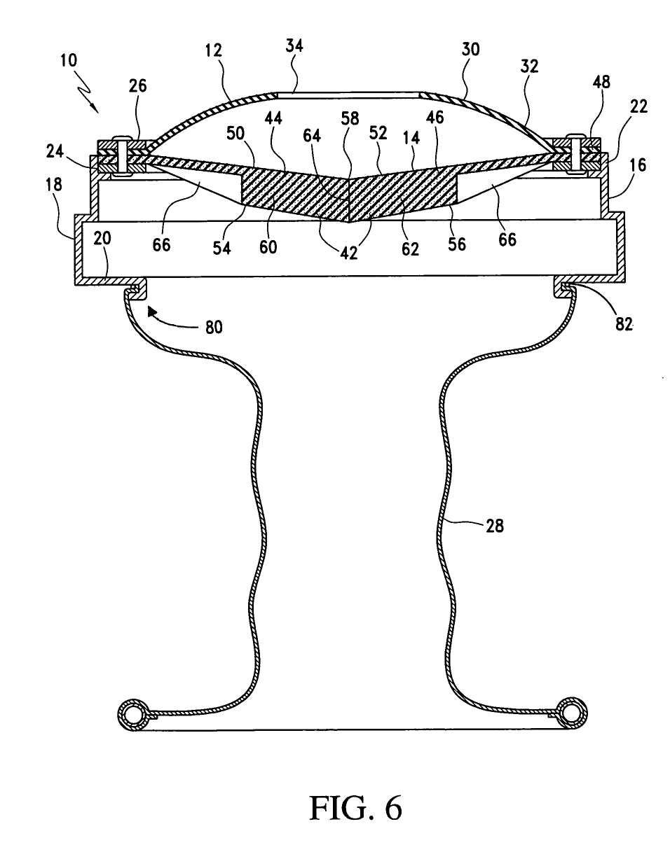

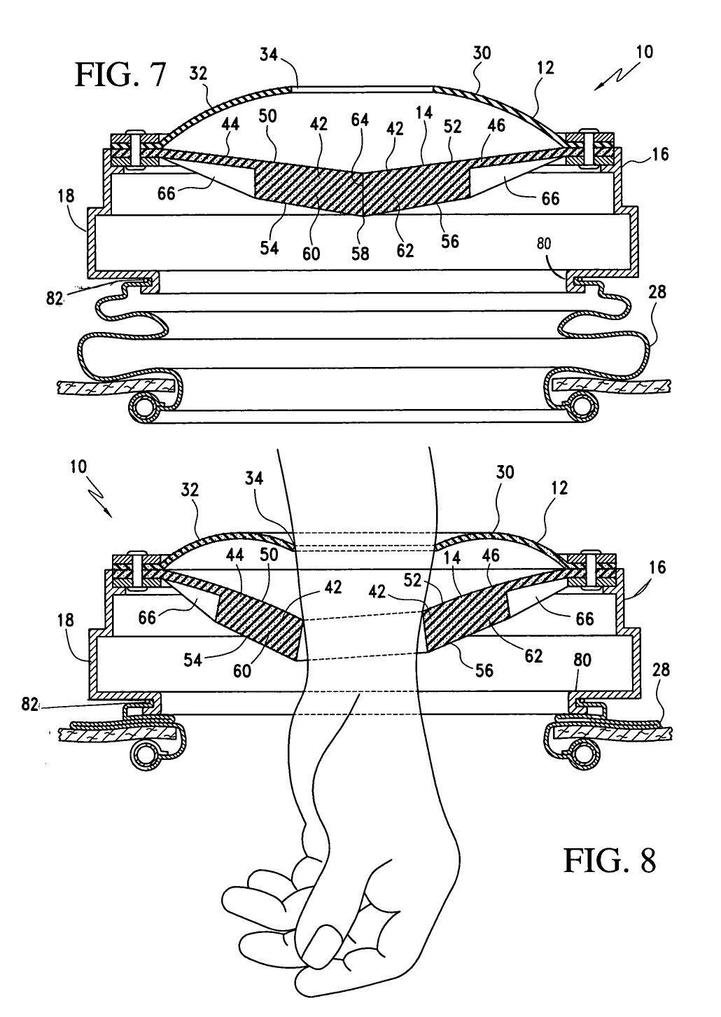

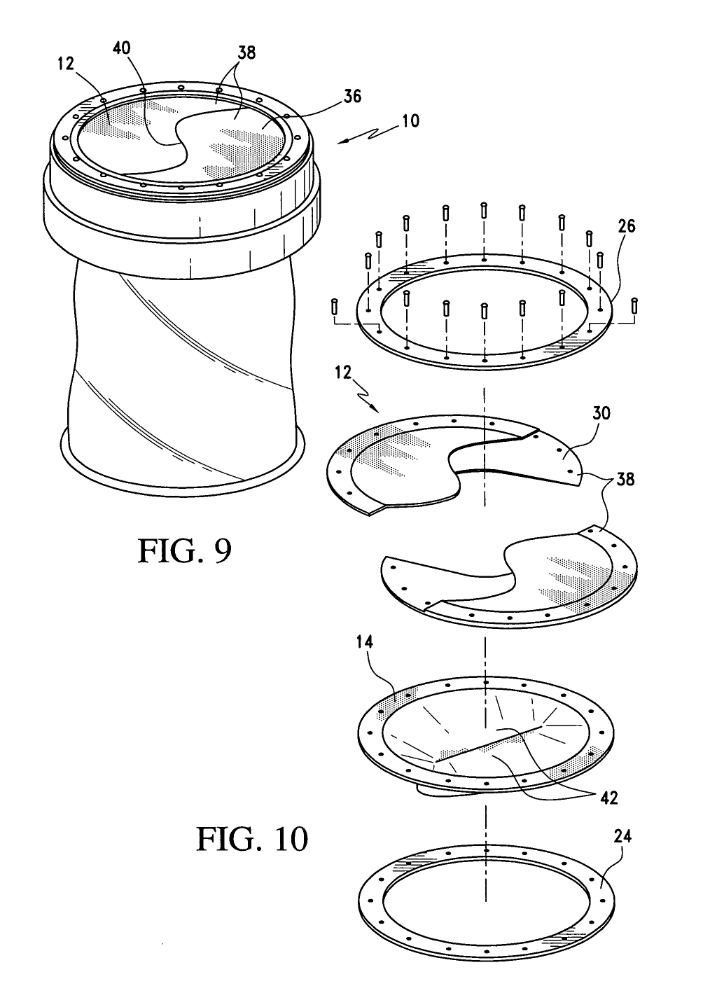

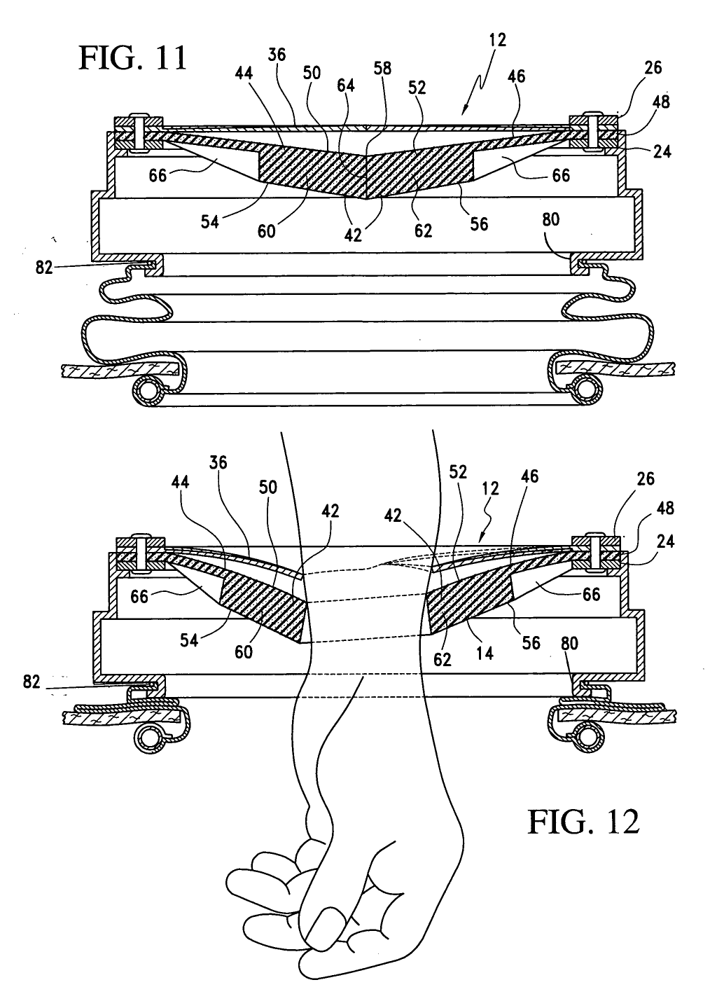

Referring to FIGS. 1 to 12, a seal apparatus 10 for permitting hand assisted laparoscopic procedures is disclosed. The seal apparatus 10 generally employs a two-seal design to ensure abdominal pressure is not compromised during hand exchanges while hand assisted laparoscopic procedures are performed. As such, the seal apparatus 10 includes an outer seal 12 and an inner seal 14. Although a two-seal design is disclosed in accordance with a preferred embodiment of the present invention, those skilled in the art will appreciate the concepts underlying the present invention may be applied to apparatuses employing additional seal layers without departing from the spirit of the present invention.

The outer seal 12 is shaped and dimensioned to create a gas tight barrier around the surgeon's wrist when inserted through the seal apparatus 10, while the inner seal 14 is shaped and dimensioned to create a gas tight barrier between the interior abdominal space and the external environment when a hand is not inserted through the seal apparatus 10.

Referring to the various figures, both the inner seal 14 and the outer seal 12 are positioned within a housing 16. In accordance with a preferred embodiment, the housing 16 is made of polyurethane, polycarbonate, and soft elastomers, such as, EPDM (Ethylene Propylene Dimonomer) and SANTOPRENE (that is, a thermoplastic elastomer), although those skilled in the art will appreciate a variety of materials may be used without departing from the spirit of the present invention. The housing 16 preferably supports the outer and inner seals 12, 14 in a concentric manner, although noncentric constructions may be employed without departing from the spirit of the present invention. More particularly, the housing 16 includes an annular base member 18. The annular base member 18 includes an inwardly directed rim 20 and a circumferentially extending outer border 22. Positioned along the inner rim 20 is a lower gasket member 24 upon which is placed the inner seal 14. Positioned directly upon the inner seal 14 is the outer seal 12, and an upper closure gasket 26 is positioned thereabove. The upper closure gasket 26 is secured to the outer seal 12, inner seal 14 and lower gasket member 24 in a manner securing all components of the present apparatus together. In accordance with a preferred embodiment, the various components are held together using mechanical attachment structures, such as, press fit rivets, adhesive, welds, or other suitable securing mechanism known to those skilled in the art.

The housing 16 is secured to the skin of an individual by first creating an incision and positioning the housing 16 adjacent the incision. Thereafter, a retractor (or wound protector) 28, which is coupled to the housing 16 at housing end 80 by an O-ring 82, is inserted into the body cavity. The skirt 29 of the retractor 28 is then drawn about the lower end of the housing 16 with the skin therebetween. As such, the housing 16 is securely supported on the skin with the retractor 28 resiliently holding the skin therebetween. Although a fixed length retractor is disclosed in accordance with a preferred embodiment of the present invention, it is contemplated adjustable retractors may be used without departing from the spirit of the present invention. For example, an adjustable retractor may be employed which allows for one to “roll-up” the free end of the retractor, or otherwise adjust the length of the retractor, for proper positioning of the present seal apparatus relative to an incision.

More particularly, and in accordance with one technique for the performance of hand assisted laparoscopic procedures, the surgical site is prepared in accordance with conventional standard hospital procedures, making sure the skin is clean and dry. Thereafter, a template is placed over the incision site and an incision line is marked upon the template using a sterile skin marker. As those skilled in the art will appreciate, the glove size dictates the size of the incision. For example, if the surgeon's glove size is 7, a 7 cm incision is usually appropriate. Thereafter, an incision is made along the marked incision line. The incision size is thereafter verified by inserting the surgeon's hand into the abdomen prior to attaching the present seal apparatus 10. If the incision is too small, the incision is extended as required on each end to maintain the central position of the incision relative to the placement of the present seal apparatus 10. Thereafter, the retractor 28 is inserted through the incision. Using one's fingers, the retractor 28 is seated evenly under the peritoneum and the area is swept to ensure that tissue is not trapped between the retractor 28 and the abdominal cavity. Thereafter, the housing 16 is pulled down into contact with the skin with the patient's tissue between the retractor 28 and the housing 16.

In accordance with a preferred embodiment, it is contemplated the outer seal 12 may be constructed with either a lip seal design or an overlapping seal design (respectively see FIGS. 1-8 and 9-12, where similar numerals are used for similar elements). However, and as those skilled in the art will certainly appreciate, other similar seal constructions, for example, an iris seal, may be employed without departing from the spirit of the present invention.

Where a lip seal 30 is employed, the lip seal 30 is shaped and dimensioned to seal about the surgeon's wrist thereby creating a gas tight barrier thereabout when inserted through the seal apparatus 10. With this in mind, the lip seal 30 includes a flexible annular membrane 32 with a central opening 34. In accordance with a preferred embodiment, the central opening 34 is approximately 2.0 inches or less, and more preferably between approximately 1.25 inches to approximately 1.5 inches. The size is chosen to comfortably accommodate a wide range of wrist and forearm sizes ensuring that most doctors will be readily able to insert their hand and wrist/forearm through the central opening 34 while allowing the central opening 34 to return to its unstressed configuration wrapped about the wrist of the doctor. The lip seal 30 is manufactured of a material allowing for the creation of contact pressure about the wrist sufficient to form a gas tight barrier without undue pressure upon the user's wrist. As such, it is contemplated the lip seal 30 will be composed of polyisoprene, urethane, neoprene, silicone, or similar materials offering desirable resilience and comfort.

Where an overlapping seal 36 is contemplated, the overlapping seal 36 is composed of multiple seal members 38 positioned about the central opening 40 in a manner creating an opening as the various seal members are pushed downwardly as one's hand is pushed therethrough. As those skilled in the art will appreciate, the various seal members 38 making up the overlapping seal 36 are shaped and dimensioned in a manner allowing them to be coupled together to create an annular space similar to the lip seal 30. However, because multiple seal members 38 are used in the construction of an overlapping seal 36, and the seal members 38 are fixedly coupled along the peripheral edge of the overlapping seal 36, the central opening 40 readily flexes to permit passage of a hand therethrough. As such, the overlapping seal 36 will open as a surgeon forces his wrist through the present seal apparatus 10 and the resiliency of the various seal members 38 will close upon the surgeon's wrist as he passes through the outer seal 12. With this in mind, and as with the lip seal construction, the overlapping seal 36 will be composed of polyisoprene, urethane, neoprene, silicone or similar materials offering desirable resilience and comfort.

While both a lip seal 30 and an overlapping seal 36 are contemplated in accordance with preferred embodiments of the present invention, it is contemplated an overlapping seal 36 will offer advantages over the lip seal 30. In particular, the contact pressure on the surgeon's wrist will be less when an overlapping seal 36 is utilized (although the gas tight barrier will be equivalent). In addition, an overlapping seal 36 will permit adjustment of the opening size to accommodate various wrist sizes and instrument sealing.

Although a lip seal and an overlapping seal are disclosed above in accordance with a preferred embodiment, those skilled in the art will appreciate various seal designs may be employed without departing from the spirit of the present invention. For example, an iris seal may be employed. In addition, it is contemplated layered seal designs may be employed within the spirit of the present invention. Specifically, a layered design might employ multiple overlapping seals as discussed above placed upon each other with their opening (and seal members) angularly offset to enhance the seals' ability to create a barrier when an individual's hand is passed therethrough.

With regard to the inner seal 14, it is preferably a duckbill seal design. Generally, the duckbill inner seal 14 includes a pair of opposed valve members 42 which open and close in much the same manner a duck opens and closes its bill. In accordance with a preferred embodiment of the present invention, the duckbill inner seal 14 includes first and second seal bodies 44, 46 extending from a circumferential flange member 48 shaped and dimensioned for mounting within the housing 16. Each of the first and second seal bodies 44, 46 includes an upper surface 50, 52 and a lower surface 54, 56. The upper surface 50, 52 is substantially smooth and consistently flat as it extends from the perimeter of the inner seal 14 to the central slit 58 where the first and second seal bodies 44, 46 meet. As will be discussed below, the lower surfaces 54, 56 of the respective first and second seal bodies 44, 46 include a thickened section 60, 62 adjacent the meeting point of the first and second seal bodies 44, 46.

The first and second seal bodies 44, 46 are mounted within the housing 16 for movement as a surgeon's hand and/or an instrument is passed therethrough. With this in mind, the proximal end of each of the first and second seal bodies 44, 46 is coupled to the housing 16 via the circumferential flange member 48, while the distal ends of the first and second seal bodies 44, 46 intersect to define an abutment face 64. The abutment face 64 is generally positioned within the center of the housing 16, and in line with the outer seal 12, to permit the passage of a hand and/or instrument therethrough. In the absence of the passage of a hand and/or instrument therethrough, the abutment face 64 is closed via the resilience of the first and second seal bodies 44, 46 as they are biased under the pressure generated from the body cavity in which the present seal apparatus 10 is positioned, for example, biased under the pressure from the abdominal insufflation gas pressure. This pressure causes the duckbill inner seal 14 to move to a closed position with the distal ends of the first and second seal bodies 44, 46 in contact.

As briefly mentioned above, the first and second seal bodies 44, 46 are formed with a thickened section 60, 62 adjacent the abutment face 64. In particular, the thickness of the first and second seal bodies 44, 46 is approximately 0.540 inch thick along the abutment face 64, while the remainder of the first and second seal bodies 44, 46 is preferably less than approximately 0.1 inch thick, and more preferably approximately 0.080 inch thick. Each of the first and second seal bodies 44, 46 is further provided with a plurality of radially extending reinforcement ribs 66 extending between the distal end of the seal bodies 44, 46 and the thickened section 60, 62 adjacent the abutment face 64.

In accordance with a preferred embodiment of the present invention, the inner seal is constructed to offer a soft yet durable construction. With this in mind, and in accordance with a preferred embodiment, the seal bodies are composed of open cell foam, for example, polyurethane, urethane-ether or polyolefin, impregnated with silicone. The seal bodies are manufactured by casting the open cell foam within a mold and then pouring silicone therein to produce seal bodies which exhibit desirable softness with durability as one passes his or her hand therethrough. As those skilled in the art will appreciate, the thickened sections 60, 62 of the first and second seal bodies 44, 46 are manufactured from silicone (or other elastomer materials, such as, urethane) reinforced with polyurethane foam while the remainder of the seal bodies 44, 46 is composed of silicone (or other elastomer materials, such as, urethane). In particular, the first and second seal bodies 44, 46 are made entirely from silicone and the thickened sections 60, 62 thereof are reinforced with polyurethane open cell foam. In addition, it is contemplated the duckbill portion may include fabric or open cell lattice structure as a reinforcement. This will provide durability while maintaining flexibility. As with the open cell foam employed in accordance with a preferred embodiment, the silicone (or other elastomer) can be combined with the reinforcement to fill the lattice structure or spaces.

In accordance with a preferred embodiment, the wall angle of the upper surface 50, 52 of the first and second seal bodies 44, 46 is maintained at an angle of approximately 30 degrees to approximately 45 degrees so as to balance the ease of passage through the inner seal 14 with the seal's ability to fully close when not in use.

It is further contemplated forces between the duckbill seal and a hand and/or instrument passing therethrough may be further minimized by adjusting wall thickness, rib geometry, surface coatings and material properties.

The positioning of the lip or overlapping seal as the outer seal and the duckbill seal as the inner seal is disclosed in accordance with a preferred embodiment. However, those skilled in the art will certainly appreciate the seal types may be reversed without adversely affecting the functionality of the present seal apparatus.

While the preferred embodiments have been shown and described, it will be understood that there is no intent to limit the invention by such disclosure, but rather, is intended to cover all modifications and alternate constructions falling within the spirit and scope of the invention.

1. A hand assisted laparoscopic seal apparatus, comprising:
a first seal;
a second seal; and
a housing supporting the first seal relative to the second seal;
wherein one of the first seal or the second seal is shaped and dimensioned for engaging and sealing around an arm of a medical practitioner and the other seal is shaped and dimensioned for sealing a body cavity from an external environment.

2. The seal apparatus according to claim 1, wherein the first seal is a lip seal having a central opening.

3. The seal apparatus according to claim 2, wherein the second seal is a duckbill seal.

4. The seal apparatus according to claim 3, wherein the first seal is an outer seal and the second seal is an inner seal.

5. The seal apparatus according to claim 2, wherein the lip seal includes a flexible annular membrane with the central opening formed therein.

6. The seal apparatus according to claim 5, wherein the central opening is approximately 2.00 inches or less.

7. The seal apparatus according to claim 5, wherein the central opening ranges between approximately 1.25 inches to approximately 1.5 inches.

8. The seal apparatus according to claim 1, wherein the first seal is an overlapping seal having a central opening.

9. The seal apparatus according to claim 8, wherein the second seal is a duckbill seal.

10. The seal apparatus according to claim 9, wherein the first seal is an outer seal and the second seal is an inner seal.

11. The seal apparatus according to claim 1, wherein the second seal is a duckbill seal.

12. The seal apparatus according to claim 11, wherein the second seal includes first and second seal bodies, each of the first and second seal bodies includes an upper surface and a lower surface, and the respective first and second seal bodies include a thickened section adjacent a meeting point of the first and second seal bodies.

13. The seal apparatus according to claim 12, wherein the thickness of the first and second seal bodies is approximately 0.540 inch along the thickened section.

14. The seal apparatus according to claim 13, wherein the first and second seal bodies are approximately less than 0.10 inch thick along a remainder thereof.

15. A hand assisted laparoscopic seal apparatus, comprising:
a seal and a housing supporting the seal, wherein the seal is composed of open cell foam impregnated with silicone.

16. The seal apparatus according to claim 15, wherein the seal is manufactured by casting the open cell foam within a mold and then pouring silicone therein to produce seal bodies exhibiting softness and durability.

17. The seal apparatus according to claim 16, wherein the open cell foam is polyurethane, urethane-ether or polyolefin.

18. The seal apparatus according to claim 17, wherein the seal is a duckbill seal.

19. The seal apparatus according to claim 18, wherein the seal includes first and second seal bodies, each of the first and second seal bodies includes an upper surface and a lower surface, and wherein the respective first and second seal bodies include a thickened section adjacent a meeting point of the first and second seal bodies.

20. The seal apparatus according to claim 19, wherein the thickness of the first and second seal bodies is approximately 0.540 inch along the thickened section.
